(19) United States
(12) Patent Application Publication
(10) Pub. No.: US 2022/0040345 A1
(43) Pub. Date: Feb. 10, 2022

(54) APPARATUS AND METHOD FOR PREVENTING BIOFOULING IN HVAC DRAIN SYSTEMS

(71) Applicant: Hatch Transformers, Inc., Tampa, FL (US)

(72) Inventors: Michael Hatch, Tampa, FL (US); John Fisher, Tampa, FL (US)

(21) Appl. No.: 17/396,573

(22) Filed: Aug. 6, 2021

Related U.S. Application Data
(60) Provisional application No. 63/063,164, filed on Aug. 7, 2020.

(51) Int. Cl. A61L 2/10; C02F 1/32

(52) U.S. Cl. CPC A61L 2/10; C02F 1/325; C02F 2201/326; A61L 2202/14; C02F 2201/3227; C02F 2303/20; A61L 2202/11; C02F 2201/3222

(57) ABSTRACT

The invention provides a solution for biofouling in HVAC condensate drain systems, including drain pans and drain lines, using ultraviolet (UV) radiation. Embodiments of the invention include one or more of: one or more ultraviolet (UV) radiation sources, positioning mechanisms for the UV radiation sources, and one or more UV radiation source control systems. Specific location of the UV radiation source control system(s) can be proximal to the UV radiation sources, or located remotely.

### FIELD OF THE INVENTION

This invention generally relates to cooling systems, and more particularly to HVAC systems with drain pan and/or condensate drain lines, and an apparatus and method for preventing biofouling of same.

### BACKGROUND OF THE INVENTION

Heating, ventilation and air conditioning (HVAC) systems provide conditioned air spaces for occupation in residential, commercial and industrial applications. One such system consists of three main components—an air handler consisting of an evaporator coil and blower, a compressor system that functions as a heat exchanger, and ductwork for transporting conditioned air throughout the occupied space. The air handler and the compressor portions of the system are connected by electrical control wiring and also by hydrofluorocarbon cooling fluid filled pipes.

During a cooling cycle the coolant in a gaseous state is compressed and cooled to a liquid state then expanded in the evaporator coils back into a gaseous state. This produces a lower temperature on the surface of the evaporator. Warm, moist air is drawn through the evaporator. As the warm moist air passes across the surface of the evaporator coil fins, the air is cooled and moisture in the warm air condenses on the cooling fins.

The moisture that is removed from the warm air collects in the drain pan area of the air handler and must be removed from the system. Typically, this is done through a drain line that is connected to the drain pan. Because there is a negative air pressure in the drain line of the drain pan, a curved ‘p-trap’ fitting is used in the drain line so that standing water in the p-trap will create enough head pressure to prevent air ingress into the system.

Because of the warm moist environment of the drain pan, biofilms and algae are prone to grow and as a result the system is subject to biofouling. Biofouling can occur in the drain pan and also in the drain pan line, including but not limited to the p-trap. If sufficient growth occurs it can cause malfunction of the drain line which in turn can cause moisture buildup in the drain pan. If not detected significant system and structural damage can occur.

Proper system design, installation practice and JHA codes typically require a safety cutoff switch that detects a rising water level in the drain pan that interrupts power to the system in the event of a faulty drain line, thereby preventing system or structural damage from occurring. However, this results in a temporary loss of functionality of the system and also requires a maintenance action to restore the drain line to its proper operating condition.

Current methods of prevention of biofoul in the drain systems include chemicals in liquid or pill form added to the drain pan or chemicals in liquid form introduced in the drain lines. These methods are temporary and must be repeated regularly to be effective.

Systems and methods of preventing biofouling in the drain pan and the drain line that requires limited or no maintenance are needed.

The invention provides such a method and corresponding assemblies. These and other advantages of the invention, as well as additional inventive features, will be apparent from the description of the invention provided herein.

### BRIEF SUMMARY OF THE INVENTION

The invention provides a solution for biofouling in HVAC condensate drain systems, including drain pans and drain lines, using ultraviolet (UV) radiation. Embodiments of the invention include one or more of: one or more ultraviolet (UV) radiation sources, positioning mechanisms for the UV radiation sources, and one or more UV radiation source control systems. Specific location of the UV radiation source control system(s) can be proximal to the UV radiation sources, or located remotely.

In an example embodiment a UV radiation source comprises one or more solid state UV emitters mounted with a flexible positioning arm, and connected to a UV light source control system. The flexible arm comprises affixing features allowing the flexible arm to be attached to the HVAC drain pan in close proximity to the drain pan's condensate exit aperture. Such affixing mechanism can comprise mounting holes or feet integral to the positioning mechanism, an adhesive system, or a mechanical clamping system, as examples. The flexible arm system including the solid state UV emitters is connected to the control system. The flexible arm system allows optimum positioning and directivity of the UV radiation.

An example embodiment comprises one or more low pressure mercury/UV fluorescent lamps, accompanied by an appropriate control system, configured and attached to the drain pan or other part of the overall system such that partial or complete coverage of the drain pan area, including drain line exit aperture, is accomplished. The control system can be proximal to the low pressure mercury/UV fluorescent lamps, or remotely located.

In an example embodiment, solid state UV emitters are interconnected, e.g., in parallel or in series, in semi-rigid string or tape form. The UV emitters are connected to the control system and can be inserted into the P-trap either from the cleanout tube or directly through the condensate exit aperture. The length of the string or tape can be such that the entire length of the P-trap includes the UV emitter tape or string. The control system can be proximal to the UV fluorescent lamps, or remotely located.

In an example embodiment a UV emitter array is inserted into a p-trap that is at the end of the drain line, usually external to the building.

In an example embodiment a semi-rigid tape or string UV array is attached on or to the entire interior perimeter edge of the drain pan to provide complete coverage of the interior drain pan surface area.

In an example embodiment, both a drain pan UV emitter system and a condensate drain line emitter system are combined to reduce or eliminate biofouling at both the drain pan and the p-trap areas of the condensate drain line.

Example embodiments provide a control system that converts an input electrical energy source to a stable, suitable power source for UV emitters, based on sensor data and control signal inputs.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1A shows a plan view of a simplified condenser/air handler section of a typical split HVAC system, including condenser, fan blower, drain pan and drain line.

FIG. 1B shows a side view of same condenser/air handler. FIG. 1B also shows an articulated view of the P-trap, including clean-out tube.

FIG. 2 shows an articulated view of the side view of the condenser/air handler but with biofoul in the p-trap are and the drain pan exit aperture.

FIGS. 3A and 3B depict illustrative embodiments of the UV emitter arrays.

FIGS. 4A and 4B show illustrative embodiments of the invention as introduced through the cleanout tube/float switch tube into and through the P-trap.

FIGS. 5A and 5B illustrate a cross-sectional view of the condensate tube and cross-section of the LED radiation pattern for the semi-rigid LED tape and the LED string.

FIG. 6 depicts a control system that controls the UV emitter devices based on sensor input information

### DETAILED DESCRIPTION OF THE INVENTION

During normal operation cooling systems use a well-understood process for controlling climate conditions in a living space. The process includes circulating the living space air through a system that simultaneously removes moisture and cools and filters the air prior to re-introducing it to the living space.

The process for conditioning the airspace includes using the thermodynamic properties of compression and expansion coupled with the liquid and gaseous state of a heat transport fluid. When the heat transport fluid is converted from a liquid to a gaseous state, the temperature of the heat transport fluid drops. If the cooled transport fluid, now in a gaseous or semi-gaseous state, is passed through a heat transfer device(s), warm moist air can be passed over the heat transfer device to simultaneously cool the air while, through the process of dew point condensation, moisture is removed from the air. This process underlies many HVAC systems.

The moisture in the air is removed through the process of condensation on the cool surface of the heat transfer device(s). The typical system design accommodates the accumulation of moisture by allowing it to drip into a collecting drain pan of which is subsequently connected to one or more drain lines. Because of the negative air pressure in drain lines, warm moist air could be drawn in to the conditioning air flow. To overcome this, a P-trap is typically created in the drain system. The presence of condensate in the P-trap offsets the negative air pressure thereby blocking warm moist air from entering the air flow of the conditioned space.

As a result of the moisture that collects in the drain pan, around the connection between the drain pan and the drain line, and at other junctures in the drain line including the P-trap area, biofouling is fostered. When biofouling occurs, the drain line can become obstructed resulting in system down time, or possibly system and building structural damage.

Ultraviolet light has the potential to deter or eliminate biofouling organisms. Studies have shown that marine biofouling can be prevented with appropriate doses of UV lighting. In U.S. Pat. No. 5,308,505, Titus, et al., report that 4000 uW/cm2 with an exposure time of 1 minute can prevent biofouling. Similarly, in US 2017/0343287, Salters indicates that 90% of a certain micro-organism can be killed with a dose as little as 10 mW-hours/m2.

Light emitting diodes can be produced in the UV spectra, and can be configured on a printed circuit board substrate, in tape and in string forms. U.S. Pat. No. 7,553,456, Gaska, et al., discloses the use of UV radiation for preventing organism growth in an HVAC system. Gaska describes a UV light source matrix for producing UV radiation. Gaska further describes a control system for his invention, and illustrates an example based on air flow areas.

The inventions described by Gaska and others do not properly address drain system components. Drain system components are subject to standing water and a warm, biofouling-conducive environment. Biofouling can occur at the drain pan exit aperture and at the interface between the drain pan and the drain line. Further, biofouling can occur in the drain line wherever moisture exists. As the biofoul further develops, more moisture can collect, thereby further fostering growth. This is also especially observed in the p-trap area of the drain line.

P-traps are designed to eliminate negative pressure unconditioned ambient air from being introduced, or drawn into, the HVAC system. As a result, they are specifically designed to continuously retain water. This also is a very conducive environment for biofoul to develop and over time and left un-serviced clog the drain line and cause system malfunction. It should be noted that P-traps can be proximally located to either the drain pan itself or the terminal point of the drain line, typically located exterior to the building and near ground level.

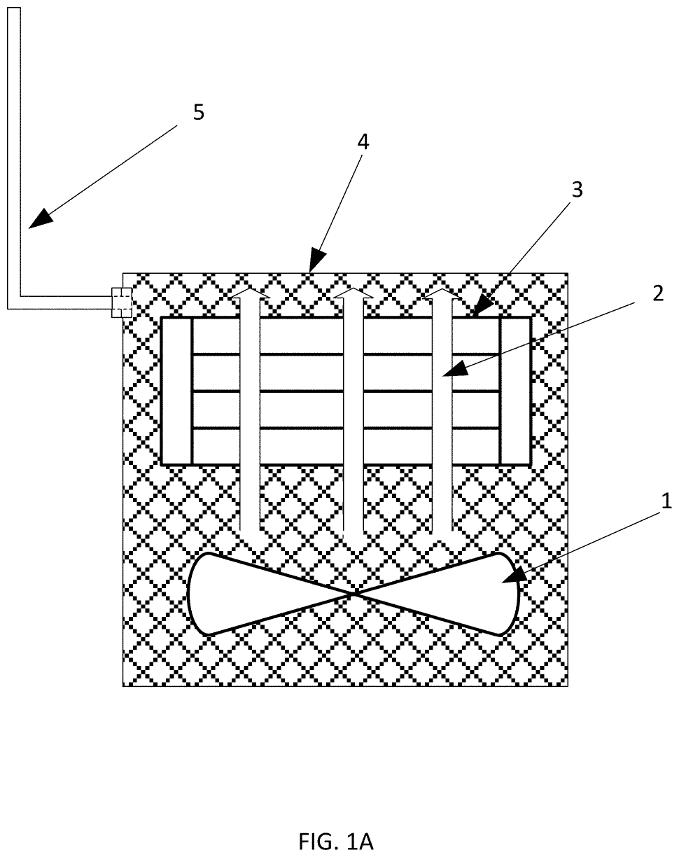

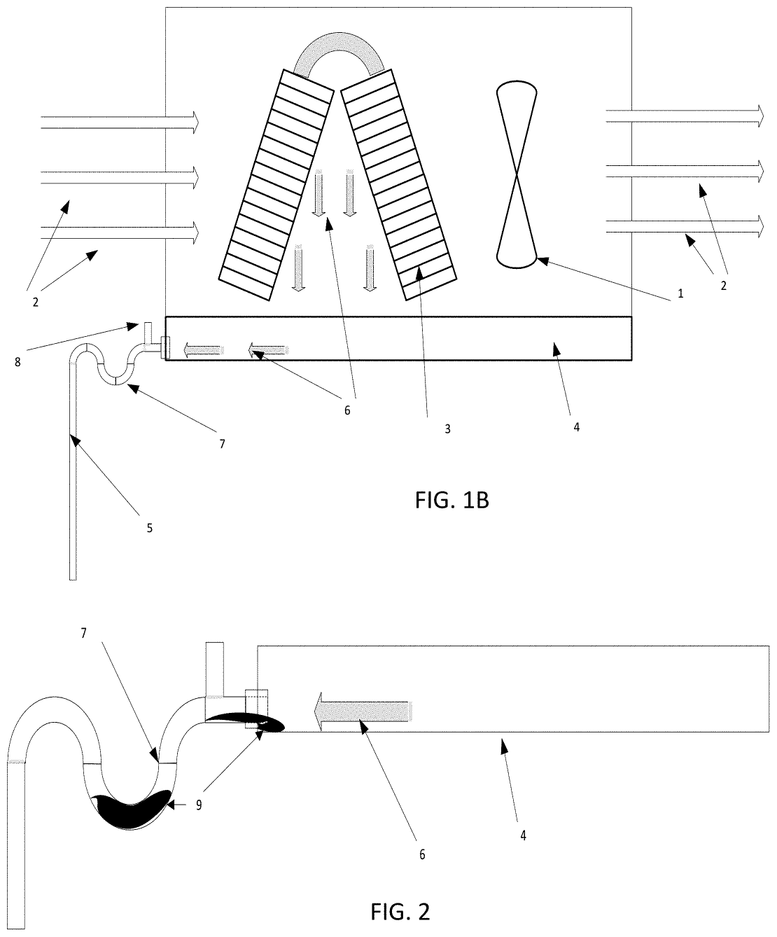

Referring to the drawings, FIGS. 1A and 1B show basic components of a typical HVAC air handler system, including a fan (1), an indication of air flow (2), a chilling coil (3), a drain pan (4) and a drain line (5). More specifically, FIG. 1B articulates moisture (6) that collects as a result of condensation. Further, FIG. 1B illustrates detailed features of drain line (5) including a proximally located p-trap (7) and an associated clean-out tube (8).

FIG. 2 illustrates the problem, where biofoul (9) is shown to develop at the drain pan (4) exit aperture and/or in the standing-water portion of the p-trap (7).

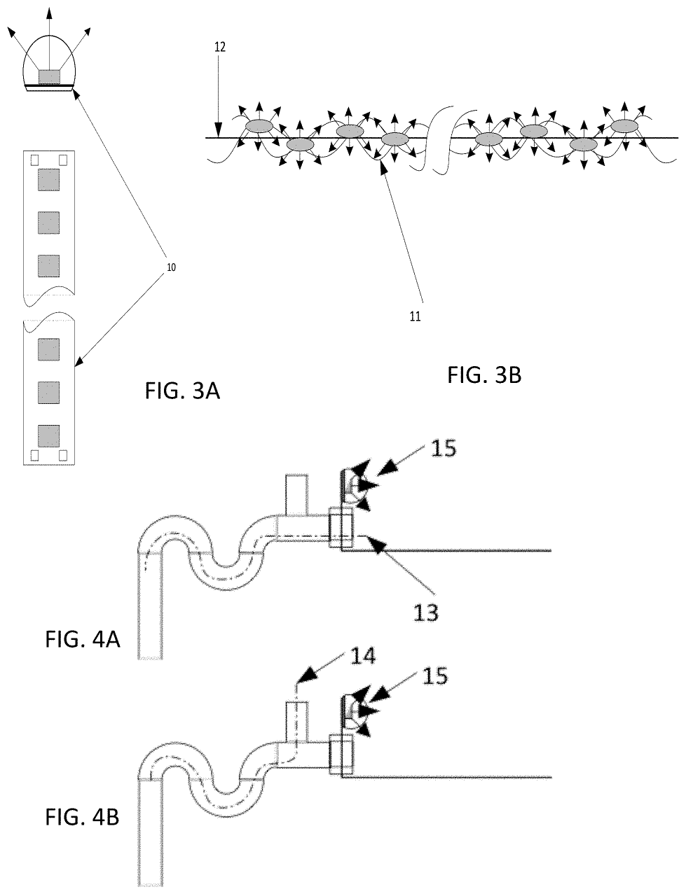

FIG. 3A and FIG. 3B illustrate two different types of linear UV radiation emitter devices, preferentially using UV light emitting diodes (LED). In FIG. 3A, the UV LED array is configured in a semi-rigid planar format, of arbitrary length (10). In FIG. 3B the UV LED array is configured in string form, that is, generally mounted directly to wires rather than a semi-rigid semi-planer substrate, of arbitrary length (11). To enhance the utility of the string form configuration a semi-rigid spline (12) can be included.

For applications in moist environments, example embodiments of both types of UV LED arrays in FIG. 3A and FIG. 3B can include moisture protection methods as needed, such as encapsulation or surface coating, preferentially in a flexible, optically clear medium.

UV-LEDs are generally classified by wavelength range, generally as shown in Table 1:

| UV | Wavelength |
|---|---|
| Classification | Range |
| UV-A | 315-400 |
| UV-B | 280-315 |
| UV-C | 100-280 |

Generally, the antibiofouling effectiveness increases as the wavelength decreases. However, the cost per milliwatt of radiation increases as the wavelength decreases. It is can be commercially advantageous in some embodiments to utilize UV-A LEDs. As such, example embodiments are described for the use of UV-A, UV-B and UV-C LED light strips.

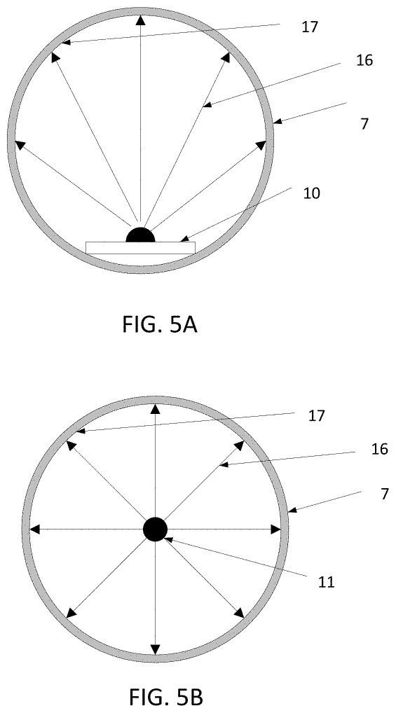

P-trap construction is typically PVC, with an inner diameter between ½″-1″. For complete internal traversal of the length of the p-trap, both prior to and through the curvature of the trap, the equivalent linear distance is commonly approximately 12 inches, although this length can vary based on diameter of the p-trap pipe and length of pipe segment from the drain pan exit aperture to the p-trap. To obtain the required radiation coverage to prevent biofouling in an example embodiment, an optical incident power is needed of about 0.5-2.0 mW/cm², or 1 mW/cm² continuous, or 2-8 mW/cm², or 4 mW/cm², pulsed at 25% duty cycle. Assuming uniform radial coverage within a 1″ internal diameter p-trap pipe with a 125 degree viewing angle UV LED, with a normalized intensity of 60% at 60 degrees, a UV LED with a radiant output power between 10 mW and 1200 mW can be used, depending on the spacing. In an example, spacing between LEDs is set such that overlap of the radiation pattern exists to provide continuous coverage. The spacing between emitters can be between 1″ and 4″. FIGS. 5A and 5B show a cross-sectional view of the p-trap (7), with either the semi-rigid tape emitter (10) or the string emitter (11). Note that emitted UV rays (16) are incident on the inner surface (17) of the p-trap, providing UV radiation to the inner surface.

FIGS. 4A and 4B illustrate example embodiments of the invention wherein the UV array is deployed to prevent biofoul in the drain system. In FIG. 4A the array (13) is inserted through the drain pan exit aperture. In FIG. 4B the array (14) is introduced through the cleanout tube. In either case the semi-rigid nature of the array facilitates traversing the distance of the drain tube. In both embodiments, either form of array may be used. In an example embodiment of the invention, the UV array (15) can be attached to the interior edge of the drain pan, circumferentially traversing a portion of or the complete interior edge of the drain pain.

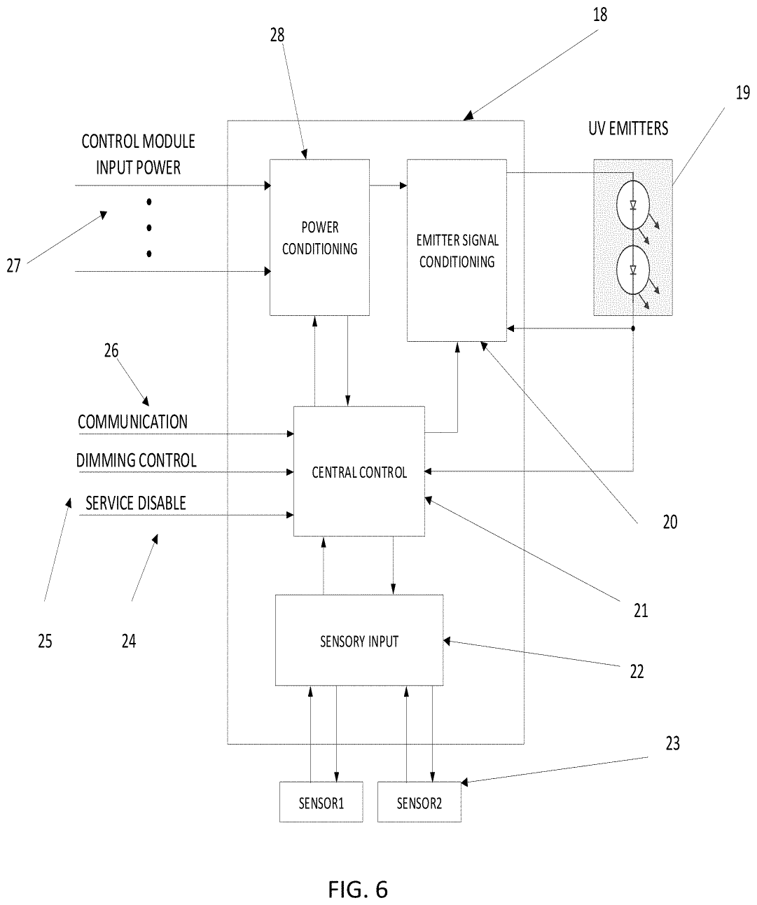

Further, in either configuration, a control module that provides electrical energy to the UV radiation sources can be connected to the array and located either adjacent to, or remote from the array. An example control module is depicted in FIG. 6. The control module (18) includes but is not limited to a power conditioning stage (28), an emitter signal conditioning stage (20), a central control section (21) and a sensory input stage (22).

The power conditioning stage (28) converts input power (27) that is supplied to the control module to a stable supply voltage for the rest of the module. The input to the power conditioning stage may consist of any numerous types of electrical levels. As examples, electrical levels covered by the invention include 12 VAC, 12 VDC, 24 VAC, 24 VDC, 36 VAC, 36 VDC, 48 VAC, 48 VDC, 120 VAC, 240 VAC or 277 VAC although the described invention and the input power stage can function using one or more of these inputs as well as other arbitrary electrical input signal levels.

The emitter signal conditioning stage (20) is configured to convert the output of the power conditioning stage (28) to the appropriate drive signal(s) for the UV emitters (19). For LED UV emitters, drive signals can comprise constant voltage or constant current signals such that forward-biasing of the UV LEDs can be controlled. Typical output voltage levels for semi-rigid LED tapes are 12 VDC, 24 VDC, 36 VDC and 48 VDC. The invention is not limited to these specific voltages. Typical rms output current values are 100 mA, 350 mA, 700 mA, 1050 mA although the invention is not limited to these specific current values. Likewise, for the invention as described for HVAC anti-biofouling, the example wattage range of the control module should be between 10 W and 100 W although the invention is not limited to this output power range.

Alternately, the signal conditioning stage (20) can be configured to control low pressure mercury UV emitters.

The sensory input stage (22) receives input from proximal or remote sensors (23). Sensors can include but are not limited to system sensing such as moisture, temperature, timing, biofoul threshold detection and A/C system power on/power off.

The central control stage (21) assimilates inputs from sensors (23) and based on algorithmic results, controls the output level of the signal conditioning stage (20). Because of the nature of UV LED emitters (19) that the control can be ON, OFF, or disabled, or operating in a dimmed condition, such that the rms level of current, or the rms level of output voltage could be controlled, thereby operating the system only in the necessary conditions for biofoul control rather than in a continuous full-on condition.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An apparatus for the control of biofoul in an HVAC system, comprising one or more UV sources disposed along a flexible base extending for a length at least equal to the length of fluid in a drain p-trap of the HVAC system, and a control system configured to supply power to the UV sources.

2. The apparatus of claim 1, wherein the flexible base comprises a tape.

3. The apparatus of claim 1, wherein the UV sources comprise UV LEDs.

4. The apparatus of claim 3, wherein the UV LEDSs are configured to provide 0.5-2.0 mW/cm² continuous, or 1 mW/cm² continuous, or 2-8 mW/cm² pulsed at 25% duty cycle, or 4 mW/cm², pulsed at 25% duty cycle.

5. The apparatus of claim 3, wherein the UV LEDs comprise 125 degree viewing angle UV LEDs, with a normalized intensity of 60% at 60 degrees, and a radiant output power between 10 mW and 1200 mW.

6. The apparatus of claim 3, wherein the UV LEDs are spaced not less than 1″ and not more than 4″ apart.

7. The apparatus of claim 1, further comprising a sensor producing a signal representative of biofoul, and wherein the control system is configured to provide power to the UV sources responsive to the sensor signal.

8. The apparatus of claim 1, wherein the UV sources comprise low pressure mercury UV emitters.

9. The apparatus of claim 1, further comprising an HVAC system having a P-trap in which the flexible base is disposed.

10. An HVAC system having a drain pan and comprising one or more UV emitters disposed on the interior perimeter edge of the drain pan.

11. The HVAC system of claim 10 wherein the one or more UV emitters are disposed such that they provide complete coverage of the interior drain pan surface area.

12. The HVAC system of claim 10, wherein the one or more UV emitters comprise UV LEDs.

13. The HVAC system of claim 10, wherein the one of more UV emitters comprise low pressure mercury UV emitters.

14. The HVAC system of claim 10, further comprising a control system configured to supply power to the UV LEDs.

15. The HVAC system of claim 14, further comprising a sensor producing a signal representative of biofoul in the HVAC system, and wherein the control system is configured to provide power to the UV emitters responsive to the sensor signal.

16. A method of reducing biofoul in a HVAC system having a drain system comprising a p-trap connected to a drain pan exit aperture or a cleanout aperture, or both, comprising providing an apparatus as in claim 1, and introducing the apparatus through the drain pan exit aperture, the cleanout aperture, or both, into the p-trap.
